(19) United States
(12) Patent Application Publication
(10) Pub. No.: US 2026/0273485 A1
(43) Pub. Date: Sep. 17, 2026

(54) SYSTEM FOR BLENDING A FRAGRANCE

(71) Applicant: SoftBank Group Corp., Tokyo (JP)

(72) Inventor: Ibuki ARITA, Tokyo (JP)

(21) Appl. No.: 19/562,775

(22) Filed: Mar. 11, 2026

Related U.S. Application Data
(60) Provisional application No. 63/770,425, filed on Mar. 12, 2025.

(51) Int. Cl. B01F 35/22; A61L 9/12; B01F 35/214; B01F 35/71; B01F 101/54; G06F 16/903

(52) U.S. Cl. CPC B01F 35/2204; A61L 9/125; B01F 35/214; B01F 35/7176; B01F 35/71805; G06F 16/90344; A61L 2209/11; A61L 2209/133; A61L 2209/134; B01F 2101/54

(57) ABSTRACT

The system includes a generative AI unit, a scent blending unit, a user interface unit, and a database unit, and receives an image of a scent from a user as input. The generative AI unit analyzes the input using natural language processing technology and extracts keywords related to the scent. The database unit stores fragrance materials corresponding to these keywords and is referred to when the AI determines the scent components.

### CROSS-REFERENCE TO RELATED APPLICATION

This application claims the benefit of priority from U.S. Provisional Patent Application No. 63/770,425, filed on Mar. 12, 2025. The entire contents of the priority application are incorporated herein by reference.

### BACKGROUND

The technology of the present disclosure relates to a system.

Japanese Unexamined Patent Publication No. 2022-180282 discloses a method, which is a persona chatbot control method performed by at least one processor, the method including a step of receiving a user utterance, a step of adding the user utterance to a prompt including an instruction sentence associated with a description regarding a character of a chatbot, a step of encoding the prompt, and a step of inputting the encoded prompt to a language model to generate a chatbot utterance responding to the user utterance.

### SUMMARY

The problem to be solved by this disclosure is the difficulty of accurately generating and providing a scent that a user desires on the spot. With conventional scent generation technologies, it has been difficult to accurately grasp what kind of scent a user specifically desires and to quickly blend a scent corresponding thereto. In particular, when a user desires a scent based on an abstract image or sensation, it is extremely complex to identify the components of the scent and combine them appropriately. Furthermore, although providing a scent in combination with other sensory experiences such as video and music is desired to obtain a deeper immersive experience, technical means for realizing this have been insufficient.

This disclosure makes it possible to accurately identify a scent desired by a user by analyzing the user's input using a generative AI and extracting keywords related to the scent. In addition, a scent blending device can generate the scent desired by the user on the spot by precisely controlling and blending fragrance materials based on instructions from the AI. Furthermore, by using video analysis technology to blend and release a scent imagined from a video in real time, it is possible to provide an immersive experience with both sight and smell. In this way, the disclosure aims to provide a scent experience that meets the diverse needs of users and to solve the problems that conventional technologies have had.

A means for solving the problem of this disclosure is to provide a system including a generative AI unit, a scent blending unit, a user interface unit, and a database unit. The generative AI unit analyzes an image of a scent input from a user and extracts related scent keywords using natural language processing technology. This analysis makes it possible to identify specific elements of the scent desired by the user. The scent blending unit precisely controls and blends a plurality of fragrance materials based on instructions from the generative AI unit to generate a specified scent. This blending unit can finely adjust the type and amount of fragrance materials and can reproduce the scent according to the user's request on the spot.

The user interface unit provides a means for a user to input an image of a scent and accepts text or voice input. This interface allows the user to intuitively convey the image of the scent to the system. The database unit stores correspondence relationships between keywords related to scents and fragrance materials, and is referred to when the generative AI unit determines the components of the scent. This database comprehensively provides information necessary for scent blending and plays a role in enhancing the analysis accuracy of the AI.

By coordinating these components, it becomes possible to quickly and accurately generate and provide the scent desired by the user. This system can solve the difficulty of identifying and blending scents that conventional technologies have had, and provide a new scent experience to the user.

### BRIEF DESCRIPTION OF DRAWINGS

FIG. 1 is a conceptual diagram illustrating an example of a configuration of a data processing system according to a first embodiment.

FIG. 2 is a conceptual diagram illustrating an example of main functions of a data processing apparatus and a smart device according to the first embodiment.

FIG. 3 is a conceptual diagram illustrating an example of a configuration of a data processing system according to a second embodiment.

FIG. 4 is a conceptual diagram illustrating an example of main functions of a data processing apparatus and smart glasses according to the second embodiment.

FIG. 5 is a conceptual diagram illustrating an example of a configuration of a data processing system according to a third embodiment.

FIG. 6 is a conceptual diagram illustrating an example of main functions of a data processing apparatus and a headset-type terminal according to the third embodiment.

FIG. 7 is a conceptual diagram illustrating an example of a configuration of a data processing system according to a fourth embodiment.

FIG. 8 is a conceptual diagram illustrating an example of main functions of a data processing apparatus and a robot according to the fourth embodiment.

FIG. 9 illustrates an emotion map on which a plurality of emotions are mapped.

FIG. 10 illustrates an emotion map on which a plurality of emotions are mapped.

FIG. 11 is a flowchart illustrating an example of a method for blending a fragrance.

### DETAILED DESCRIPTION

Hereinafter, an example of an embodiment of a system according to the technology of the present disclosure will be described with reference to the accompanying drawings.

First, terms used in the following description will be described.

In the following embodiments, a processor with a reference sign (hereinafter, simply referred to as a “processor”) may be one arithmetic device or may be a combination of a plurality of arithmetic devices. Also, the processor may be one type of arithmetic device or may be a combination of a plurality of types of arithmetic devices. Examples of the arithmetic device include a CPU (Central Processing Unit), a GPU (Graphics Processing Unit), a GPGPU (General-Purpose computing on Graphics Processing Units), an APU (Accelerated Processing Unit), and the like.

In the following embodiments, a RAM (Random Access Memory) with a reference sign is a memory in which information is temporarily stored, and is used as a work memory by a processor.

In the following embodiments, a storage with a reference sign is one or more non-volatile storage devices that store various programs, various parameters, and the like. Examples of the non-volatile storage device include a flash memory (SSD (Solid State Drive)), a magnetic disk (for example, a hard disk), or a magnetic tape, and the like.

In the following embodiments, a communication I/F (Interface) with a reference sign is an interface including a communication processor, an antenna, and the like. The communication I/F manages communication among a plurality of computers. An example of a communication standard applied to the communication I/F includes a wireless communication standard including 5G (5th Generation Mobile Communication System), Wi-Fi (registered trademark), or Bluetooth (registered trademark), and the like.

In the following embodiments, “A and/or B” is synonymous with “at least one of A and B”. That is, “A and/or B” means that it may be A only, B only, or a combination of A and B. Also, in the present specification, when three or more matters are expressed by being connected with “and/or”, the same concept as “A and/or B” is applied.

### First Embodiment

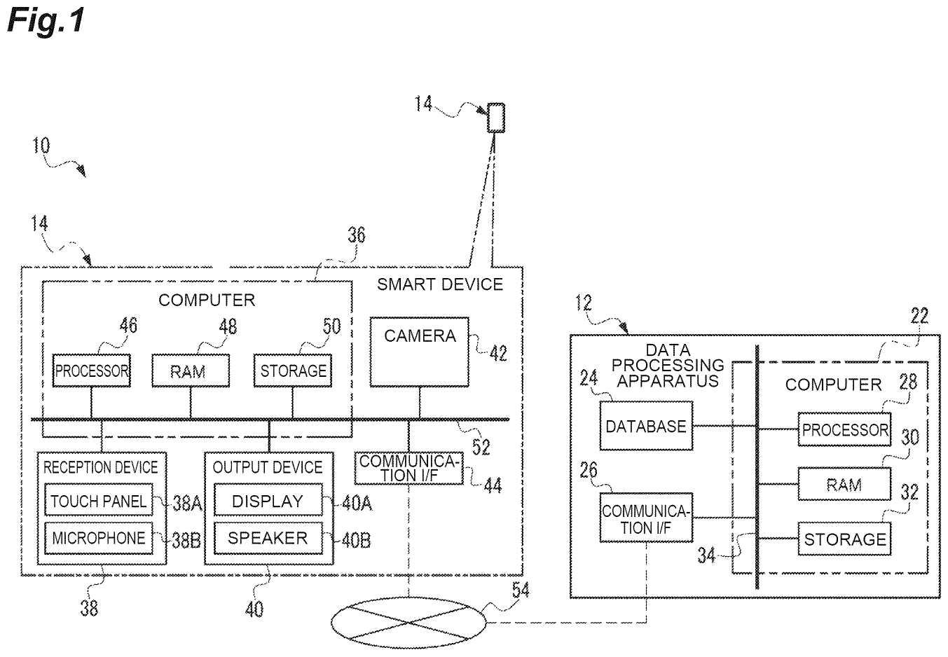

FIG. 1 illustrates an example of a configuration of a data processing system 10 according to a first embodiment.

As illustrated in FIG. 1, the data processing system 10 includes a data processing apparatus 12 and a smart device 14. An example of the data processing apparatus 12 includes a server.

The data processing apparatus 12 includes a computer 22, a database 24, and a communication I/F 26. The computer 22 is an example of a “computer” according to the technology of the present disclosure. The computer 22 includes a processor 28, a RAM 30, and a storage 32. The processor 28, the RAM 30, and the storage 32 are connected to a bus 34. Also, the database 24 and the communication I/F 26 are also connected to the bus 34. The communication I/F 26 is connected to a network 54. An example of the network 54 includes a WAN (Wide Area Network) and/or a LAN (Local Area Network), and the like.

The smart device 14 includes a computer 36, a reception device 38, an output device 40, a camera 42, and a communication I/F 44. The computer 36 includes a processor 46, a RAM 48, and a storage 50. The processor 46, the RAM 48, and the storage 50 are connected to a bus 52. Also, the reception device 38, the output device 40, and the camera 42 are also connected to the bus 52.

The reception device 38 includes a touch panel 38A, a microphone 38B, and the like, and receives a user input. The touch panel 38A receives a user input by contact of an indicator by detecting contact of the indicator (for example, a pen or a finger, etc.). The microphone 38B receives a user input by voice by detecting a user's voice. A control unit 46A transmits data indicating the user input received by the touch panel 38A and the microphone 38B to the data processing apparatus 12. In the data processing apparatus 12, a specific processing unit 290 acquires the data indicating the user input.

The output device 40 includes a display 40A, a speaker 40B, and the like, and presents data to a user 20 by outputting the data in a representation form (for example, voice and/or text) perceivable by the user 20. The display 40A displays visible information such as text and images in accordance with an instruction from the processor 46. The speaker 40B outputs voice in accordance with an instruction from the processor 46. The camera 42 is a small digital camera on which an optical system such as a lens, a diaphragm, and a shutter, and an image sensor such as a CMOS (Complementary Metal-Oxide-Semiconductor) image sensor or a CCD (Charge Coupled Device) image sensor are mounted.

The communication I/F 44 is connected to the network 54. The communication I/Fs 44 and 26 manage exchange of various information between the processor 46 and the processor 28 via the network 54.

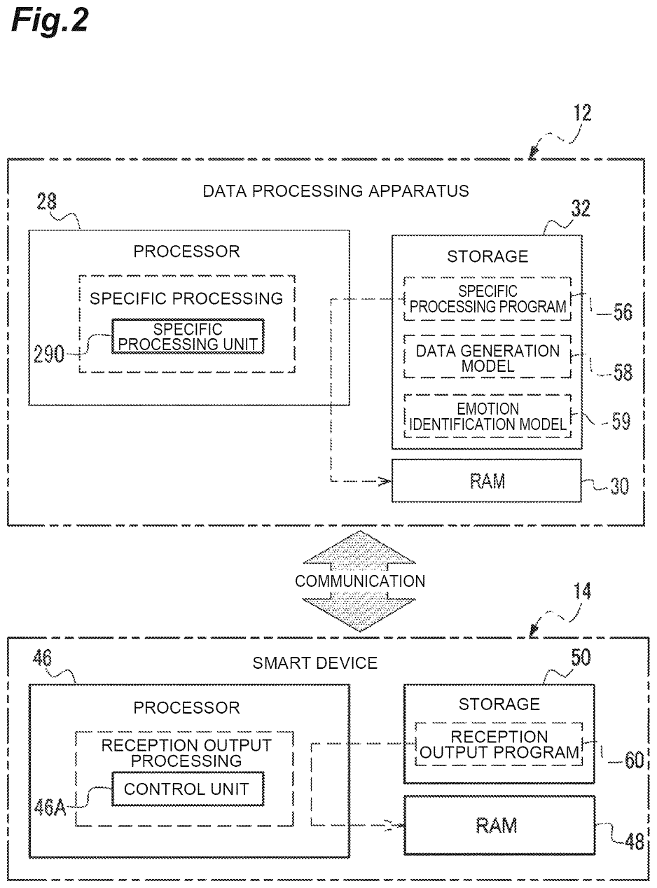

FIG. 2 illustrates an example of main functions of the data processing apparatus 12 and the smart device 14.

As illustrated in FIG. 2, in the data processing apparatus 12, specific processing is performed by the processor 28. A specific processing program 56 is stored in the storage 32. The specific processing program 56 is an example of a “program” according to the technology of the present disclosure. The processor 28 reads the specific processing program 56 from the storage 32 and executes the read specific processing program 56 on the RAM 30. The specific processing is realized by the processor 28 operating as a specific processing unit 290 in accordance with the specific processing program 56 executed on the RAM 30.

A data generation model 58 and an emotion identification model 59 are stored in the storage 32. The data generation model 58 and the emotion identification model 59 are used by the specific processing unit 290. The specific processing unit 290 can estimate a user's emotion using the emotion identification model 59 and perform specific processing using the user's emotion. In an emotion estimation function (emotion identification function) using the emotion identification model 59, various estimations and predictions regarding the user's emotion, including estimation and prediction of the user's emotion, are performed, but the present disclosure is not limited to such an example. Also, the estimation and prediction of emotion include, for example, analysis (analytics) of emotion and the like.

In the smart device 14, reception output processing is performed by the processor 46. A reception output program 60 is stored in the storage 50. The reception output program 60 is used in combination with the specific processing program 56 by the data processing system 10. The processor 46 reads the reception output program 60 from the storage 50 and executes the read reception output program 60 on the RAM 48. The specific processing is realized by the processor 46 operating as a control unit 46A in accordance with the specific processing program 60 executed on the RAM 48. Note that the smart device 14 can also have a data generation model and an emotion identification model similar to the data generation model 58 and the emotion identification model 59, and perform processing similar to that of the specific processing unit 290 using these models. The reception output processing is realized by the processor 46 operating as the control unit 46A in accordance with the reception output program 60 executed on the RAM 48.

Note that an apparatus other than the data processing apparatus 12 may have the data generation model 58. For example, a server apparatus (for example, a generation server) may have the data generation model 58. In this case, the data processing apparatus 12 obtains a processing result (such as a prediction result) in which the data generation model 58 is used, by communicating with the server apparatus having the data generation model 58. Also, the data processing apparatus 12 may be a server apparatus, or may be a terminal device owned by a user (for example, a mobile phone, a robot, a home appliance, etc.). Next, an example of processing by the data processing system 10 according to the first embodiment will be described.

### Example 1

A flow of specific processing in Example 1 will be described. Each unit of the system described below is realized by the data processing apparatus 12 and the smart device 14. Also, the data processing apparatus 12 is referred to as a “server”, and the smart device 14 is referred to as a “terminal”.

### Embodiment

Details of a case where an embodiment is realized using a server and a terminal will be described more specifically. This system includes a generative AI unit, a scent blending unit, a user interface unit, and a database unit in order to generate and provide a scent desired by a user.

First, the generative AI unit is realized on a server. The generative AI unit may be configured by, for example, the specific processing unit 290, the specific processing program 56, and the data generation model 58. This unit receives input from a user and performs analysis using natural language processing technology. When the user inputs “a refreshing scent in a forest,” the generative AI unit extracts keywords such as “wood,” “leaves,” “moisture,” and “soil.” This analysis uses advanced natural language processing algorithms that run on the server, making it possible to process vast amounts of data quickly. Specifically, the AI breaks down the input text and identifies keywords related to scents by checking the meaning of each word against a dictionary database. The generative AI unit also has a function to analyze video data, and for example, when a scene of the sea is displayed, it recognizes elements such as “salt,” “water,” “algae,” and “sand,” and imagines a related scent. This video analysis uses image recognition technology to identify objects in the video and extract scent elements based on them.

Natural language processing and keyword extraction in the generative AI unit may be executed based on calculations of semantic proximity in a high-dimensional vector space, rather than mere phrase replacement. Specifically, the generative AI unit may include a neural network based on a Transformer architecture (e.g., a BERT or GPT-based model), tokenize input text data, and convert each token into a multidimensional vector (embedding representation). The database unit may similarly store characteristics of each fragrance (note, volatility, emotional effect) in vectorized form. The generative AI unit may calculate cosine similarity between the vector of the input text and vectors in the fragrance database, and select fragrances having a similarity exceeding a predetermined threshold as candidates. This vector calculation processing is performed using a model with millions of parameters, processing at a computational volume and speed that is practically impossible to perform by human mental activity.

Furthermore, the generative AI unit may have a regression model for determining the blending ratio of each fragrance based on the extracted keywords. This regression model is trained using past blending data and user feedback (evaluation of pleasantness/unpleasantness) as training data, predicts optimal scent intensity and longevity for the input image, and may calculate the discharge amount of each fragrance in milligrams or as a duty ratio of pulse width modulation (PWM).

Analysis of video data by the generative AI unit may be implemented by performing semantic segmentation for each video frame using a Convolutional Neural Network (CNN) or Vision Transformer (ViT). The system may sample images from a video stream at predetermined intervals (e.g., 1 frame per second) and quantify not only objects in the image (sea, flowers, bonfire, etc.) but also the color tone of the scene (warm colors, cool colors) and lighting conditions. Furthermore, by using a multimodal learning model (e.g., CLIP), image features and text (scent descriptor) features may be mapped to the same embedding space to directly infer scent components from the visual information of the video. This makes it possible to synchronize appropriate scents in real time even for abstract video expressions that are difficult to verbalize.

Next, the scent blending unit is realized on a terminal. The scent blending unit may be configured by, for example, the computer 36 or the output device 40. This unit precisely controls and blends a plurality of fragrance materials based on instructions from the generative AI unit. The terminal is equipped with fragrance material cartridges for example in the output device 40, and the output device 40 includes pumps and valves for adjusting the type and amount of each fragrance material. Although not shown in FIG. 1, the output device 40 may include the fragrance material cartridges 40C, the pumps 40D, and the valves 40E. For example, when a user requests “the scent of fresh oranges,” the scent blending unit releases an appropriate amount of orange fragrance material and blends it with other fragrance materials. This process is performed in real time, allowing the user to smell the scent on the spot. The scent blending unit is provided with precision sensors and control circuits to accurately measure minute amounts of fragrance materials, thereby making it possible to enhance the reproducibility of the scent.

The scent blending unit may include a microcontroller (MCU) that converts digital command values received from the generative AI unit into physical control signals. Specifically, the MCU may perform PID control (Proportional-Integral-Derivative control) on piezoelectric pumps or solenoid valves connected to each fragrance cartridge to achieve the specified blending ratio. This corrects for variations in discharge amount due to fragrance viscosity, remaining amount, and ambient temperature changes, minimizing errors between the theoretical blending ratio calculated by the AI and the physical composition of the actually released scent. This feedback control is executed in loops on the order of milliseconds, achieving precision and response speed impossible for human manual blending.

Additionally, the scent blending unit may be equipped downstream with a gas sensor (e.g., a metal oxide semiconductor sensor or a quartz crystal microbalance sensor) for detecting the concentration of released scent components. The gas sensor may be connected to, for example, the bus 52 and positioned alongside the reception device 38, the output device 40, etc. The MCU may monitor detection values from the gas sensor in real time, calculate the difference (error) between the target scent profile and the actual detection values, and immediately adjust the drive voltage of the pumps. This closed-loop control system provides the technical effect of maintaining a constant scent experience perceived by the user.

The system may further execute a resource management algorithm for optimizing fragrance consumption. The generative AI unit and the scent blending unit may refer to remaining amount data of currently available fragrance cartridges when generating a scent desired by a user. If a specific fragrance is insufficient, the generative AI unit and the scent blending unit may search for alternative fragrances having similar scent characteristics in the vector space, or combinations of a plurality of other fragrances (alternative blends), and dynamically change blending instructions to generate a scent within available resources without impairing the user's requested scent image. This processing solves the technical problem specific to computer systems of preventing service interruption due to fragrance depletion and improving system operating efficiency.

The user interface unit is realized on the terminal and provides a means for the user to input an image of a scent. The user interface unit may be configured by, for example, the reception device 38. This interface can accept text input or voice input, allowing the user to intuitively interact with the system. For example, when a user gives a voice instruction “I want the scent of a rose,” the interface unit converts the voice into text and transmits it to the generative AI unit. This voice recognition uses a voice analysis algorithm, and noise removal and accurate text conversion of the voice are performed.

The database unit is realized on the server and stores correspondence relationships between keywords related to scents and fragrance materials. The database unit may be configured by, for example, the database 24 or the storage 32. This database is referred to when the generative AI unit determines the components of the scent. For example, for the keyword “wood,” fragrance materials such as cedarwood and pine correspond, and for the keyword “flower,” rose and jasmine correspond. The database comprehensively provides information necessary for scent blending and plays a role in enhancing the analysis accuracy of the AI. The database can also include past user preferences and trend information, thereby making it possible to provide a personalized scent experience.

In this way, by coordinating the server and the terminal, it becomes possible to quickly and accurately generate and provide the scent desired by the user. This system aims to solve the difficulty of identifying and blending scents that conventional technologies have had, and to provide a new scent experience to the user. Furthermore, the system has scalability and can always provide the latest scent experience by adding new fragrance materials and analysis algorithms.

### (System Configuration)

The system according to the present embodiment includes a generative AI unit, a scent blending unit, a user interface unit, and a database unit. The generative AI unit receives input from a user and performs analysis using natural language processing technology. Specifically, when a user inputs a prompt sentence such as “a refreshing scent in a forest,” the generative AI unit extracts keywords such as “wood,” “leaves,” “moisture,” and “soil.” This analysis uses advanced natural language processing algorithms that run on a server, making it possible to process vast amounts of data quickly. For example, the AI breaks down the input text and identifies keywords related to scents by checking the meaning of each word against a dictionary database. The generative AI unit also has a function to analyze video data, and for example, when a scene of the sea is displayed, it recognizes elements such as “salt,” “water,” “algae,” and “sand,” and imagines a related scent. This video analysis uses image recognition technology to identify objects in the video and extract scent elements based on them.

The scent blending unit precisely controls and blends a plurality of fragrance materials based on instructions from the generative AI unit. A terminal is equipped with fragrance material cartridges, and includes pumps and valves for adjusting the type and amount of each fragrance material. For example, when a user requests “the scent of fresh oranges,” the scent blending unit releases an appropriate amount of orange fragrance material and blends it with other fragrance materials. This process is performed in real time, allowing the user to smell the scent on the spot. The scent blending unit is provided with precision sensors and control circuits to accurately measure minute amounts of fragrance materials, thereby making it possible to enhance the reproducibility of the scent. Furthermore, the scent blending unit also has a function to adjust the intensity of the scent according to the user's preference, and for example, can increase the release amount of fragrance materials for a user who desires a stronger scent.

The user interface unit provides a means for a user to input an image of a scent. This interface can accept text input or voice input, allowing the user to intuitively interact with the system. For example, when a user gives a voice instruction “I want the scent of a rose,” the interface unit converts the voice into text and transmits it to the generative AI unit. This voice recognition uses a voice analysis algorithm, and noise removal and accurate text conversion of the voice are performed. Furthermore, the user interface unit also has a function to display a history of scents previously input by the user, thereby allowing the user to easily reproduce a scent tried before.

The database unit stores correspondence relationships between keywords related to scents and fragrance materials. This database is referred to when the generative AI unit determines the components of the scent. For example, for the keyword “wood,” fragrance materials such as cedarwood and pine correspond, and for the keyword “flower,” rose and jasmine correspond. The database comprehensively provides information necessary for scent blending and plays a role in enhancing the analysis accuracy of the AI. The database can also include past user preferences and trend information, thereby making it possible to provide a personalized scent experience. Furthermore, the database also has a function to suggest scents according to the season or time of day, and for example, can suggest floral scents in spring and refreshing scents in summer.

In this way, by coordinating the server and the terminal, it becomes possible to quickly and accurately generate and provide the scent desired by the user. This system aims to solve the difficulty of identifying and blending scents that conventional technologies have had, and to provide a new scent experience to the user. Furthermore, the system has scalability and can always provide the latest scent experience by adding new fragrance materials and analysis algorithms.

### (Implementation Steps)

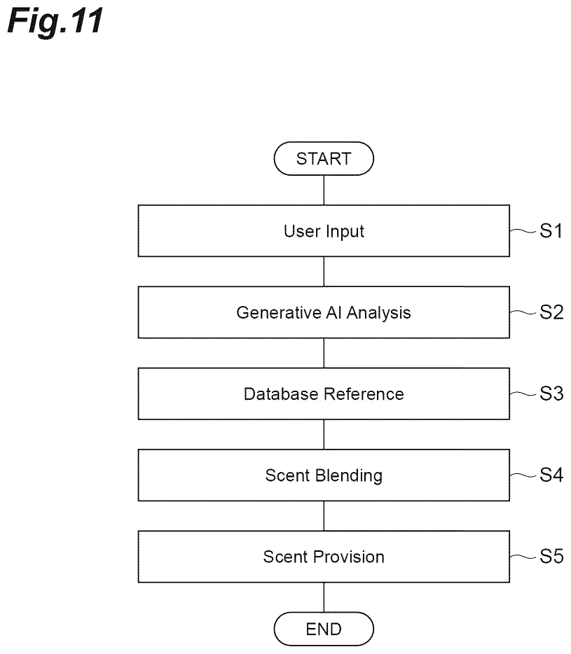

### Step 1: User Input Step. (See Step S1 in FIG. 11)

A user inputs an image of a scent they want to smell through the user interface unit of a terminal. This input can be done by text or voice. For example, the user inputs a specific image such as “a refreshing scent in a forest” or “the scent of fresh oranges.” In the case of voice input, the voice is converted into text using voice recognition technology to a format that the system can understand. The user interface unit plays a role in transmitting the input data to the generative AI unit.

### Step 2: Generative AI Analysis Step. (See Step S2 in FIG. 11)

The generative AI unit analyzes the input received from the user. It extracts keywords related to scents from the input text using natural language processing technology. For example, from the input “a refreshing scent in a forest,” keywords such as “wood,” “leaves,” “moisture,” and “soil” are extracted. A specific example of a prompt sentence to be read into the generative AI could be an instruction such as, “Analyze the image of the scent desired by the user and identify the related scent components.” The AI generates information for determining the scent components based on these keywords.

### Step 3: Database Reference Step. (See Step S3 in FIG. 11)

The generative AI unit refers to the database unit based on the analyzed keywords. The database stores correspondence relationships between keywords related to scents and fragrance materials. For example, for the keyword “wood,” fragrance materials such as cedarwood and pine correspond, and for the keyword “flower,” rose and jasmine correspond. The database provides necessary information when the AI determines the scent components and plays a role in enhancing the analysis accuracy.

### Step 4: Scent Blending Step. (See Step S4 in FIG. 11)

The scent blending unit precisely controls and blends a plurality of fragrance materials based on instructions from the generative AI unit. The terminal is equipped with fragrance material cartridges, and includes pumps and valves for adjusting the type and amount of each fragrance material. For example, when a user requests “the scent of fresh oranges,” the scent blending unit releases an appropriate amount of orange fragrance material and blends it with other fragrance materials. This process is performed in real time, allowing the user to smell the scent on the spot.

### Step 5: Scent Provision Step. (See Step S5 in FIG. 11)

The blended scent is released from the scent blending unit and provided to the user. The user can smell the generated scent on the spot and can obtain the desired scent experience. The intensity and duration of the scent are adjustable according to the user's preference, making it possible to provide a personalized scent experience. This allows the user to enjoy a new experience through the sense of smell along with sight and hearing.

### Specific Use Case

For example, suppose a user wants to relax at home and experience a scent as if they were forest bathing. In this case, the user inputs a prompt “a refreshing scent in a forest” through the user interface unit of the terminal. This prompt is transmitted to the generative AI unit, and the AI performs analysis using natural language processing technology. A specific example of a prompt sentence to be read into the generative AI could be an instruction such as, “The user is requesting a scent as if they were forest bathing. Please identify the related scent components.”

The generative AI unit extracts keywords such as “wood,” “leaves,” “moisture,” and “soil” from the input prompt. Based on these keywords, the AI refers to the database unit and determines the scent components. The database stores fragrance materials corresponding to each keyword, for example, cedarwood and pine correspond to “wood,” and eucalyptus and mint correspond to “leaves.”

Next, the scent blending unit selects the necessary fragrance materials from the fragrance material cartridges based on instructions from the generative AI unit, and performs blending with precise control. For example, it mixes cedarwood and eucalyptus fragrance materials in an appropriate ratio to reproduce the scent of the forest desired by the user. This blending is performed in real time, and the user can smell the scent on the spot.

The blended scent is released from the scent blending unit and provided to the user. By smelling the generated scent, the user can have a relaxing experience as if they were in a forest. The intensity and duration of the scent are adjustable according to the user's preference, making it possible to provide a personalized scent experience.

As described above, the present disclosure is a system that makes it possible to quickly and accurately generate and provide a specific scent desired by a user, and solves the difficulty of identifying and blending scents that conventional technologies have had.

### Application Example 1

A flow of specific processing in Application Example 1 will be described. Each unit of the system described below is realized by the data processing apparatus 12 and the smart device 14. Also, the data processing apparatus 12 is referred to as a “server”, and the smart device 14 is referred to as a “terminal”.

### Embodiment

An embodiment will be described more specifically and in detail. The present disclosure is a system that provides an immersive experience utilizing the sense of smell in addition to sight and hearing in a content distribution service. This system includes a generative AI unit, a scent blending unit, and a user interface unit, and aims to generate a scent in real time according to the content and provide it to the user.

First, the generative AI unit will be described in detail. This unit analyzes video and music data input from a content distribution platform. It uses video analysis technology to recognize scenes and objects in the video and extracts scent elements based on them. For example, when a scene of the sea is displayed in a movie, the AI recognizes elements such as “salt,” “water,” and “algae,” and imagines a related scent. This process uses an image recognition algorithm, which analyzes colors, movements, shapes, and the like in the video to identify the characteristics of the scene. It also uses music analysis technology to analyze the tempo and atmosphere of the music, and for example, associates a “lavender” or “chamomile” scent with a gentle melody, and a “citrus” or “mint” scent with an intense rhythm. Music analysis includes frequency analysis of sound waves and recognition of rhythm patterns, which makes it possible to grasp the emotional tone of the music.

The generative AI unit determines the scent components based on these analysis results and sends instructions to the scent blending unit. These instructions include the specific types of fragrance materials and their blending ratios, and provide detailed guidelines for the scent blending unit to accurately reproduce the scent.

Next, the scent blending unit will be described in detail. This unit selects the necessary fragrance materials from a plurality of fragrance material cartridges based on instructions from the generative AI unit, and performs blending with precise control. The scent blending unit includes pumps and valves for adjusting the type and amount of each fragrance material. For example, to match a sea scene, it mixes “salt” and “water” fragrance materials in an appropriate ratio to create a sense of a sea breeze and provides it to the user. This process is performed in real time, allowing the user to smell the scent on the spot. The scent blending unit is provided with precision sensors and control circuits to accurately measure minute amounts of fragrance materials, thereby making it possible to enhance the reproducibility of the scent. Furthermore, the scent blending unit also has a function to adjust the intensity of the scent according to the user's preference, and for example, can increase the release amount of fragrance materials for a user who desires a stronger scent.

The user interface unit will be described in detail. This unit provides a means for the user to adjust the intensity and duration of the scent. The user can change the scent settings through the terminal, and for example, can increase the release amount of fragrance materials when a stronger scent is desired. The user interface unit also has a function to display a history of previously experienced scents, allowing the user to easily reproduce a scent tried before. Furthermore, the user interface unit can continuously improve the scent generation process by collecting user feedback and feeding it back to the generative AI unit.

In this way, the system can generate a scent according to the content of video or music, and allow the user to experience the content through the three senses of sight, hearing, and smell. This significantly improves the immersive experience of the content, allowing the user to enter more deeply into the world of the content. This system can be applied to various content such as movies, music, and games, and can provide a new form of entertainment. Furthermore, the system has scalability and can always provide the latest scent experience by adding new fragrance materials and analysis algorithms.

### (System Configuration)

The system according to the present embodiment includes a generative AI unit, a scent blending unit, and a user interface unit. The generative AI unit analyzes video and music data input from a content distribution platform. It uses video analysis technology to recognize scenes and objects in the video and extracts scent elements based on them. For example, when a scene of the sea is displayed in a movie, the AI recognizes elements such as “salt,” “water,” and “algae,” and imagines a related scent. This process uses an image recognition algorithm, which analyzes colors, movements, shapes, and the like in the video to identify the characteristics of the scene. It also uses music analysis technology to analyze the tempo and atmosphere of the music, and for example, associates a “lavender” or “chamomile” scent with a gentle melody, and a “citrus” or “mint” scent with an intense rhythm. Music analysis includes frequency analysis of sound waves and recognition of rhythm patterns, which makes it possible to grasp the emotional tone of the music. The generative AI unit determines the scent components based on these analysis results and sends instructions to the scent blending unit. These instructions include the specific types of fragrance materials and their blending ratios, and provide detailed guidelines for the scent blending unit to accurately reproduce the scent. A specific example of a prompt sentence to be read into the generative AI could be an instruction such as, “Generate a scent for the user to be immersed in a sea scene. Identify the related scent components and create blending instructions.”

The scent blending unit selects the necessary fragrance materials from a plurality of fragrance material cartridges based on instructions from the generative AI unit, and performs blending with precise control. The scent blending unit includes pumps and valves for adjusting the type and amount of each fragrance material. For example, to match a sea scene, it mixes “salt” and “water” fragrance materials in an appropriate ratio to create a sense of a sea breeze and provides it to the user. This process is performed in real time, allowing the user to smell the scent on the spot. The scent blending unit is provided with precision sensors and control circuits to accurately measure minute amounts of fragrance materials, thereby making it possible to enhance the reproducibility of the scent. Furthermore, the scent blending unit also has a function to adjust the intensity of the scent according to the user's preference, and for example, can increase the release amount of fragrance materials for a user who desires a stronger scent. In addition, the scent blending unit is provided with an algorithm for optimizing the consumption of fragrance materials, thereby making it possible to minimize the waste of fragrance materials.

The user interface unit provides a means for the user to adjust the intensity and duration of the scent. The user can change the scent settings through the terminal, and for example, can increase the release amount of fragrance materials when a stronger scent is desired. The user interface unit also has a function to display a history of previously experienced scents, allowing the user to easily reproduce a scent tried before. Furthermore, the user interface unit can continuously improve the scent generation process by collecting user feedback and feeding it back to the generative AI unit. The user interface unit adopts a visually intuitive design and is designed so that the user can operate it easily. This allows the user to individually customize the scent experience, making it possible to provide a more personalized experience.

In this way, the system can generate a scent according to the content of video or music, and allow the user to experience the content through the three senses of sight, hearing, and smell. This significantly improves the immersive experience of the content, allowing the user to enter more deeply into the world of the content. This system can be applied to various content such as movies, music, and games, and can provide a new form of entertainment. Furthermore, the system has scalability and can always provide the latest scent experience by adding new fragrance materials and analysis algorithms.

### (Implementation Steps)

Step 1: Content Data Input Step. Video or music data is input into the system from a content distribution platform. This data is part of the content that the user is watching or listening to, and serves as the basic information for generating a scent based on the scene of the video or the atmosphere of the music. For example, if a sea scene is included in a movie, the video data is input into the system, allowing the process to proceed to the next analysis step.

Step 2: Generative AI Analysis Step. The generative AI unit analyzes the input video or music data. In this step, it uses video analysis technology to recognize scenes and objects in the video and extracts scent elements based on them. For example, in a sea scene, elements such as “salt,” “water,” and “algae” are recognized. It also uses music analysis technology to analyze the tempo and atmosphere of the music, and associates a “lavender” or “chamomile” scent with a gentle melody, and a “citrus” or “mint” scent with an intense rhythm. A specific example of a prompt sentence to be read into the generative AI could be an instruction such as, “Generate a scent for the user to be immersed in a sea scene. Identify the related scent components and create blending instructions.”

Step 3: Scent Blending Step. The scent blending unit selects the necessary fragrance materials based on instructions from the generative AI unit, and performs blending with precise control. The scent blending unit is provided with a plurality of fragrance material cartridges, and includes pumps and valves for adjusting the type and amount of each fragrance material. For example, to match a sea scene, it mixes “salt” and “water” fragrance materials in an appropriate ratio to create a sense of a sea breeze and provides it to the user. This process is performed in real time, allowing the user to smell the scent on the spot.

Step 4: Scent Provision Step. The blended scent is released from the scent blending unit and provided to the user. The user can smell the generated scent on the spot and can obtain the desired scent experience. The intensity and duration of the scent are adjustable according to the user's preference, making it possible to provide a personalized scent experience. This allows the user to enjoy a new experience through the sense of smell along with sight and hearing.

Step 5: User Interface Adjustment Step. The user interface unit provides a means for the user to adjust the intensity and duration of the scent. The user can change the scent settings through the terminal, and for example, can increase the release amount of fragrance materials when a stronger scent is desired. The user interface unit also has a function to display a history of previously experienced scents, allowing the user to easily reproduce a scent tried before. Furthermore, the user interface unit can continuously improve the scent generation process by collecting user feedback and feeding it back to the generative AI unit.

### Specific Use Case

For example, consider a case where a user wants a more immersive experience while watching a movie at home. This user wishes to enjoy a scent that matches the scenes depicted in the movie. When the movie scene shifts to a beach, the user wants to feel the sea breeze and the scent of the sea. In this case, the system analyzes the movie's video data and recognizes the sea scene. The generative AI unit extracts elements such as “salt,” “water,” and “algae” using video analysis technology and determines the scent components based on these.

A specific example of a prompt sentence to be read into the generative AI could be an instruction such as, “Generate a scent for the user to be immersed in a beach scene. Identify the related scent components and create blending instructions.” Based on this prompt, the AI selects appropriate fragrance materials from the database and sends specific instructions to the scent blending unit.

The scent blending unit follows the instructions from the AI, selects “salt” and “water” fragrance materials from the fragrance material cartridges, and mixes them in an appropriate ratio. This blending is performed in real time, allowing the user to smell the scent in sync with the movie scene. The intensity and duration of the scent are adjustable through the user interface unit, and the user can customize the scent experience according to their preference.

In this way, the user can experience the movie through the three senses of sight, hearing, and smell, and can become more deeply immersed in the world of the movie. This system can be applied not only to movies but also to various content such as music and games, and can provide a new form of entertainment. This allows the user to have a richer experience, and the appeal of the content is further enhanced.

The specific processing unit 290 transmits a result of the specific processing to the smart device 14. In the smart device 14, the control unit 46A causes the output device 40 to output the result of the specific processing. The microphone 38B acquires voice indicating a user input for the result of the specific processing. The control unit 46A transmits voice data indicating the user input acquired by the microphone 38B to the data processing apparatus 12. In the data processing apparatus 12, the specific processing unit 290 acquires the voice data.

The data generation model 58 is a so-called generative AI (Artificial Intelligence). An example of the data generation model 58 includes a generative AI such as ChatGPT (registered trademark) (Internet search <URL: https://openai.com/blog/chatgpt>). The data generation model 58 is obtained by causing a neural network to perform deep learning. A prompt including an instruction is input to the data generation model 58, and inference data such as voice data indicating voice, text data indicating text, and image data indicating an image (for example, still image data or moving image data) is input. The data generation model 58 infers the input inference data in accordance with the instruction indicated by the prompt, and outputs an inference result in one or more data formats among voice data, text data, image data, and the like. The data generation model 58 includes, for example, a text generation AI, an image generation AI, a multimodal generation AI, and the like. Here, inference refers to, for example, analysis, classification, prediction, and/or summarization, and the like. The specific processing unit 290 performs the above-described specific processing while using the data generation model 58. The data generation model 58 may be a model fine-tuned to output an inference result from a prompt that does not include an instruction, and in this case, the data generation model 58 can output an inference result from a prompt that does not include an instruction. In the data processing apparatus 12 and the like, a plurality of types of data generation models 58 are included, and the data generation model 58 includes AIs other than generative AI. Als other than generative AI are, for example, linear regression, logistic regression, a decision tree, a random forest, a support vector machine (SVM), k-means clustering, a convolutional neural network (CNN), a recurrent neural network (RNN), a generative adversarial network (GAN), or naive Bayes, and can perform various processes, but the present disclosure is not limited to such examples. Also, the AI may be an AI agent. Also, when the processing of each unit described above is performed by an AI, the processing is partially or entirely performed by the AI, but the present disclosure is not limited to such an example. Also, a process implemented by an AI including a generative AI may be replaced with a rule-based process, and a rule-based process may be replaced with a process implemented by an AI including a generative AI.

Also, the processing by the data processing system 10 described above is executed by the specific processing unit 290 of the data processing apparatus 12 or the control unit 46A of the smart device 14, but may also be executed by the specific processing unit 290 of the data processing apparatus 12 and the control unit 46A of the smart device 14. Also, the specific processing unit 290 of the data processing apparatus 12 acquires or collects information necessary for the processing from the smart device 14 or an external device, and the smart device 14 acquires or collects information necessary for the processing from the data processing apparatus 12 or an external device.

For example, a collection unit is realized by the control unit 46A of the smart device 14 or the specific processing unit 290 of the data processing apparatus 12. For example, an acquisition unit acquires step count data using the camera 42 or the communication I/F 44 of the smart device 14, and the data is processed by the specific processing unit 290 of the data processing apparatus 12. For example, an analysis unit is realized by the specific processing unit 290 of the data processing apparatus 12, and analyzes data from the collection unit and the acquisition unit. For example, a generation unit is realized by the specific processing unit 290 of the data processing apparatus 12, and generates a cooking menu using a generative AI. For example, a provision unit is realized by the output device 40 of the smart device 14 or the specific processing unit 290 of the data processing apparatus 12, and provides the generated cooking menu to a user. The correspondence relationship between each unit and the device or the control unit is not limited to the above-described example, and various changes are possible.

In the above embodiment, an example form in which the specific processing is performed by the data processing apparatus 12 has been described, but the technology of the present disclosure is not limited to this, and the specific processing may be performed by the smart device 14.

### Second Embodiment

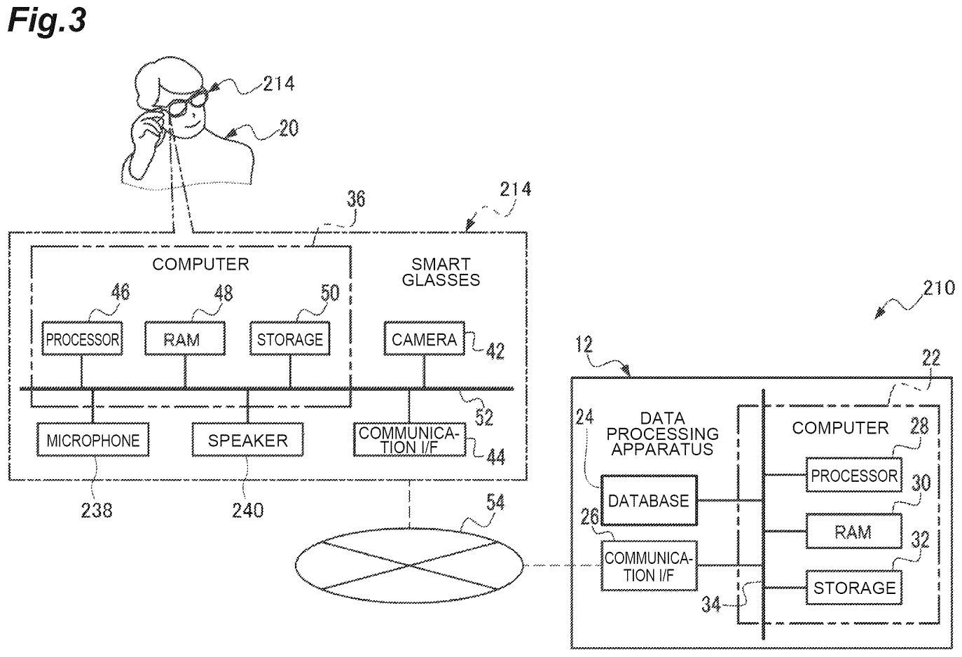

FIG. 3 illustrates an example of a configuration of a data processing system 210 according to a second embodiment.

As illustrated in FIG. 3, the data processing system 210 includes a data processing apparatus 12 and smart glasses 214. An example of the data processing apparatus 12 includes a server.

The data processing apparatus 12 includes a computer 22, a database 24, and a communication I/F 26. The computer 22 is an example of a “computer” according to the technology of the present disclosure. The computer 22 includes a processor 28, a RAM 30, and a storage 32. The processor 28, the RAM 30, and the storage 32 are connected to a bus 34. Also, the database 24 and the communication I/F 26 are also connected to the bus 34. The communication I/F 26 is connected to a network 54. An example of the network 54 includes a WAN (Wide Area Network) and/or a LAN (Local Area Network), and the like.

The smart glasses 214 include a computer 36, a microphone 238, a speaker 240, a camera 42, and a communication I/F 44. The computer 36 includes a processor 46, a RAM 48, and a storage 50. The processor 46, the RAM 48, and the storage 50 are connected to a bus 52. Also, the microphone 238, the speaker 240, and the camera 42 are also connected to the bus 52.

The microphone 238 receives an instruction or the like from a user 20 by receiving voice uttered by the user 20. The microphone 238 captures the voice uttered by the user 20, converts the captured voice into voice data, and outputs the voice data to the processor 46. The speaker 240 outputs voice in accordance with an instruction from the processor 46.

The camera 42 is a small digital camera on which an optical system such as a lens, a diaphragm, and a shutter, and an image sensor such as a CMOS (Complementary Metal-Oxide-Semiconductor) image sensor or a CCD (Charge Coupled Device) image sensor are mounted, and images the surroundings of the user 20 (for example, an imaging range defined by an angle of view corresponding to the width of the field of view of a general person with normal vision).

The communication I/F 44 is connected to the network 54. The communication I/Fs 44 and 26 manage exchange of various information between the processor 46 and the processor 28 via the network 54. The exchange of various information between the processor 46 and the processor 28 using the communication I/Fs 44 and 26 is performed in a secure state.

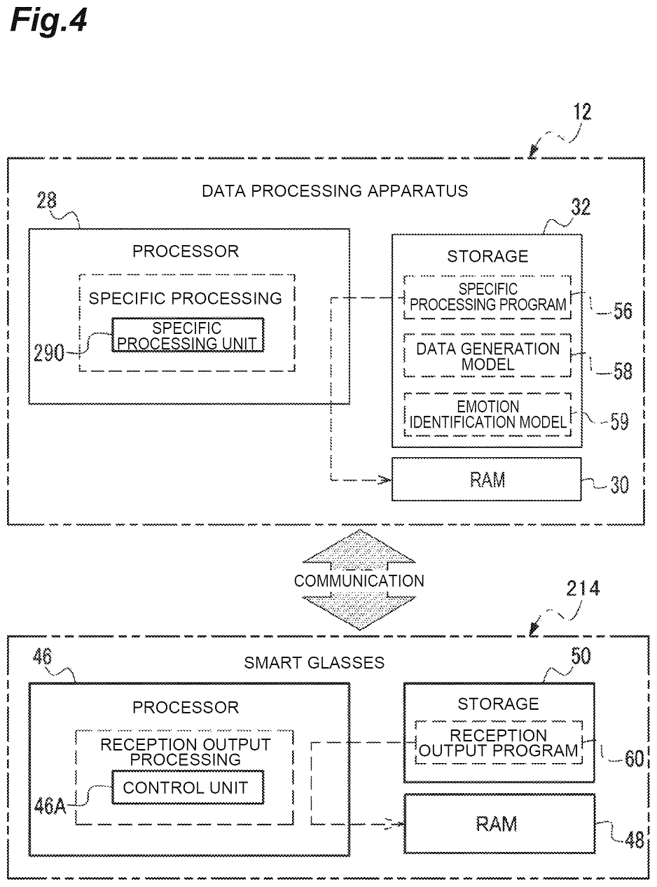

FIG. 4 illustrates an example of main functions of the data processing apparatus 12 and the smart glasses 214. As illustrated in FIG. 4, in the data processing apparatus 12, specific processing is performed by the processor 28. A specific processing program 56 is stored in the storage 32.

The specific processing program 56 is an example of a “program” according to the technology of the present disclosure. The processor 28 reads the specific processing program 56 from the storage 32 and executes the read specific processing program 56 on the RAM 30. The specific processing is realized by the processor 28 operating as a specific processing unit 290 in accordance with the specific processing program 56 executed on the RAM 30.

A data generation model 58 and an emotion identification model 59 are stored in the storage 32. The data generation model 58 and the emotion identification model 59 are used by the specific processing unit 290. The specific processing unit 290 can estimate a user's emotion using the emotion identification model 59 and perform specific processing using the user's emotion. In an emotion estimation function (emotion identification function) using the emotion identification model 59, various estimations and predictions regarding the user's emotion, including estimation and prediction of the user's emotion, are performed, but the present disclosure is not limited to such an example. Also, the estimation and prediction of emotion include, for example, analysis (analytics) of emotion and the like.

In the smart glasses 214, reception output processing is performed by the processor 46. A reception output program 60 is stored in the storage 50. The processor 46 reads the reception output program 60 from the storage 50 and executes the read reception output program 60 on the RAM 48. The reception output processing is realized by the processor 46 operating as a control unit 46A in accordance with the reception output program 60 executed on the RAM 48. Note that the smart glasses 214 can also have a data generation model and an emotion identification model similar to the data generation model 58 and the emotion identification model 59, and perform processing similar to that of the specific processing unit 290 using these models.

Next, specific processing by the specific processing unit 290 of the data processing apparatus 12 will be described. Each unit of the system described below is realized by the data processing apparatus 12 and the smart glasses 214. In the following description, the data processing apparatus 12 is referred to as a “server”, and the smart glasses 214 are referred to as a “terminal”.

### Example 1

Since the flow of the specific processing is the same as that in Example 1 described in the first embodiment, a description thereof is omitted.

### Application Example 1

Since the flow of the specific processing is the same as that in Example 1 described in the first embodiment, a description thereof is omitted.

The specific processing unit 290 transmits a result of the specific processing to the smart glasses 214. In the smart glasses 214, the control unit 46A causes the speaker 240 to output the result of the specific processing. The microphone 238 acquires voice indicating a user input for the result of the specific processing. The control unit 46A transmits voice data indicating the user input acquired by the microphone 238 to the data processing apparatus 12. In the data processing apparatus 12, the specific processing unit 290 acquires the voice data.

The data generation model 58 is a so-called generative AI (Artificial Intelligence). An example of the data generation model 58 includes a generative AI such as ChatGPT (registered trademark) (Internet search <URL: https://openai.com/blog/chatgpt>). The data generation model 58 is obtained by causing a neural network to perform deep learning. A prompt including an instruction is input to the data generation model 58, and inference data such as voice data indicating voice, text data indicating text, and image data indicating an image (for example, still image data or moving image data) is input. The data generation model 58 infers the input inference data in accordance with the instruction indicated by the prompt, and outputs an inference result in one or more data formats among voice data, text data, image data, and the like. The data generation model 58 includes, for example, a text generation AI, an image generation AI, a multimodal generation AI, and the like. Here, inference refers to, for example, analysis, classification, prediction, and/or summarization, and the like. The specific processing unit 290 performs the above-described specific processing while using the data generation model 58. The data generation model 58 may be a model fine-tuned to output an inference result from a prompt that does not include an instruction, and in this case, the data generation model 58 can output an inference result from a prompt that does not include an instruction. In the data processing apparatus 12 and the like, a plurality of types of data generation models 58 are included, and the data generation model 58 includes AIs other than generative AI. AIs other than generative AI are, for example, linear regression, logistic regression, a decision tree, a random forest, a support vector machine (SVM), k-means clustering, a convolutional neural network (CNN), a recurrent neural network (RNN), a generative adversarial network (GAN), or naive Bayes, and can perform various processes, but the present disclosure is not limited to such examples. Also, the AI may be an AI agent. Also, when the processing of each unit described above is performed by an AI, the processing is partially or entirely performed by the AI, but the present disclosure is not limited to such an example. Also, a process implemented by an AI including a generative AI may be replaced with a rule-based process, and a rule-based process may be replaced with a process implemented by an AI including a generative AI.

Also, the processing by the data processing system 10 described above is executed by the specific processing unit 290 of the data processing apparatus 12 or the control unit 46A of the smart device 14, but may also be executed by the specific processing unit 290 of the data processing apparatus 12 and the control unit 46A of the smart device 14. Also, the specific processing unit 290 of the data processing apparatus 12 acquires or collects information necessary for the processing from the smart device 14 or an external device, and the smart device 14 acquires or collects information necessary for the processing from the data processing apparatus 12 or an external device.

For example, a collection unit is realized by the control unit 46A of the smart device 14 or the specific processing unit 290 of the data processing apparatus 12. For example, an acquisition unit acquires step count data using the camera 42 or the communication I/F 44 of the smart device 14, and the data is processed by the specific processing unit 290 of the data processing apparatus 12. For example, an analysis unit is realized by the specific processing unit 290 of the data processing apparatus 12, and analyzes data from the collection unit and the acquisition unit. For example, a generation unit is realized by the specific processing unit 290 of the data processing apparatus 12, and generates a cooking menu using a generative AI. For example, a provision unit is realized by the output device 40 of the smart device 14 or the specific processing unit 290 of the data processing apparatus 12, and provides the generated cooking menu to a user. The correspondence relationship between each unit and the device or the control unit is not limited to the above-described example, and various changes are possible.

In the above embodiment, an example form in which the specific processing is performed by the data processing apparatus 12 has been described, but the technology of the present disclosure is not limited to this, and the specific processing may be performed by the smart glasses 214.

### Third Embodiment

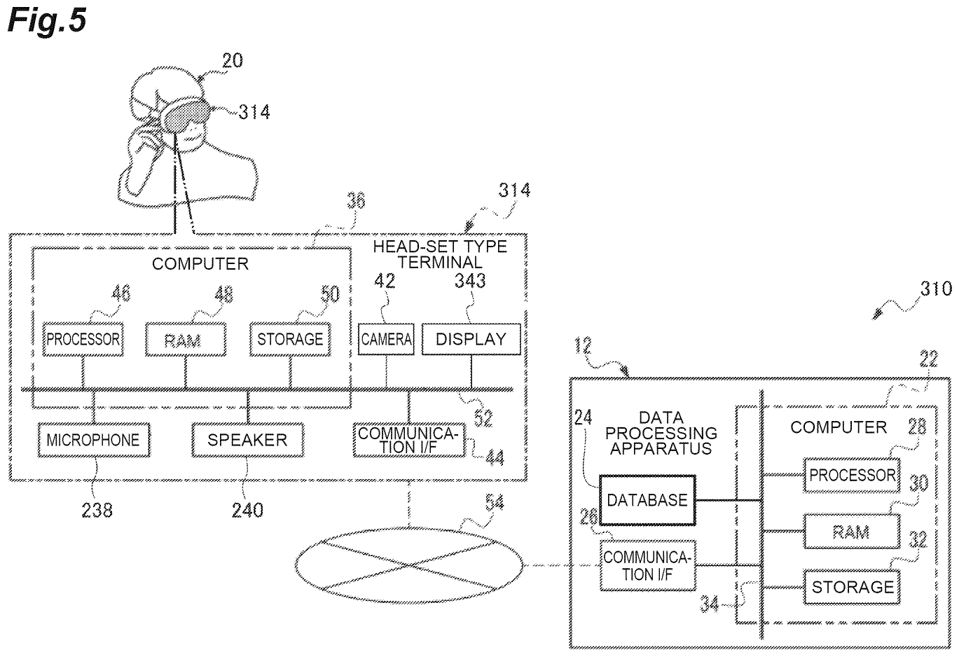

FIG. 5 illustrates an example of a configuration of a data processing system 310 according to a third embodiment.

As illustrated in FIG. 5, the data processing system 310 includes a data processing apparatus 12 and a headset-type terminal 314. An example of the data processing apparatus 12 includes a server.

The data processing apparatus 12 includes a computer 22, a database 24, and a communication I/F 26. The computer 22 is an example of a “computer” according to the technology of the present disclosure. The computer 22 includes a processor 28, a RAM 30, and a storage 32. The processor 28, the RAM 30, and the storage 32 are connected to a bus 34. Also, the database 24 and the communication I/F 26 are also connected to the bus 34. The communication I/F 26 is connected to a network 54. An example of the network 54 includes a WAN (Wide Area Network) and/or a LAN (Local Area Network), and the like.

The headset-type terminal 314 includes a computer 36, a microphone 238, a speaker 240, a camera 42, a communication I/F 44, and a display 343. The computer 36 includes a processor 46, a RAM 48, and a storage 50. The processor 46, the RAM 48, and the storage 50 are connected to a bus 52. Also, the microphone 238, the speaker 240, the camera 42, and the display 343 are also connected to the bus 52.

The microphone 238 receives an instruction or the like from a user 20 by receiving voice uttered by the user 20. The microphone 238 captures the voice uttered by the user 20, converts the captured voice into voice data, and outputs the voice data to the processor 46. The speaker 240 outputs voice in accordance with an instruction from the processor 46.

The camera 42 is a small digital camera on which an optical system such as a lens, a diaphragm, and a shutter, and an image sensor such as a CMOS (Complementary Metal-Oxide-Semiconductor) image sensor or a CCD (Charge Coupled Device) image sensor are mounted, and images the surroundings of the user 20 (for example, an imaging range defined by an angle of view corresponding to the width of the field of view of a general person with normal vision).

The communication I/F 44 is connected to the network 54. The communication I/Fs 44 and 26 manage exchange of various information between the processor 46 and the processor 28 via the network 54. The exchange of various information between the processor 46 and the processor 28 using the communication I/Fs 44 and 26 is performed in a secure state.

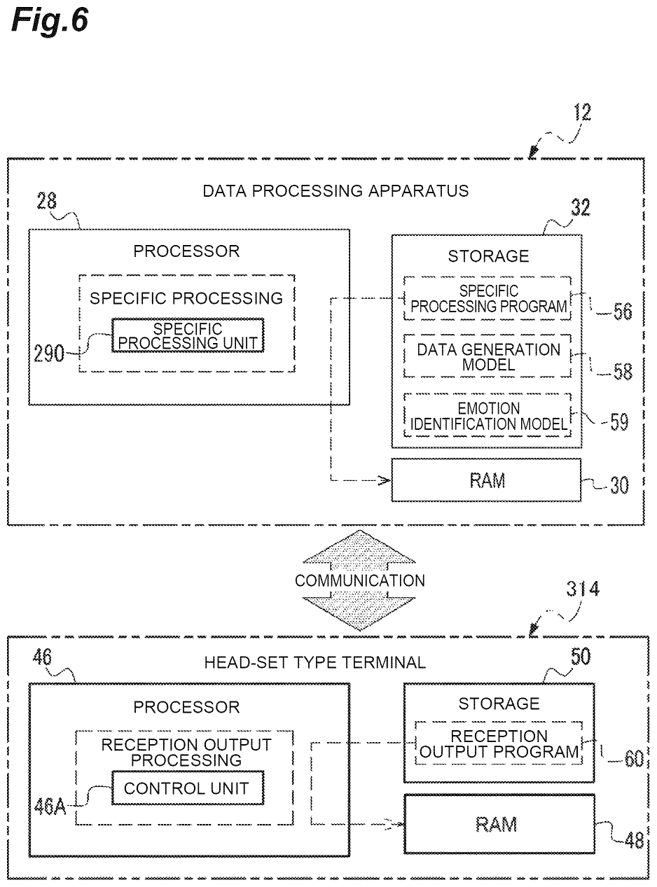

FIG. 6 illustrates an example of main functions of the data processing apparatus 12 and the headset-type terminal 314. As illustrated in FIG. 6, in the data processing apparatus 12, specific processing is performed by the processor 28. A specific processing program 56 is stored in the storage 32.

The specific processing program 56 is an example of a “program” according to the technology of the present disclosure. The processor 28 reads the specific processing program 56 from the storage 32 and executes the read specific processing program 56 on the RAM 30. The specific processing is realized by the processor 28 operating as a specific processing unit 290 in accordance with the specific processing program 56 executed on the RAM 30.

A data generation model 58 and an emotion identification model 59 are stored in the storage 32. The data generation model 58 and the emotion identification model 59 are used by the specific processing unit 290.

In the headset-type terminal 314, reception output processing is performed by the processor 46. A reception output program 60 is stored in the storage 50. The processor 46 reads the reception output program 60 from the storage 50 and executes the read reception output program 60 on the RAM 48. The reception output processing is realized by the processor 46 operating as a control unit 46A in accordance with the reception output program 60 executed on the RAM 48.

Next, specific processing by the specific processing unit 290 of the data processing apparatus 12 will be described. Each unit of the system described below is realized by the data processing apparatus 12 and the headset-type terminal 314. In the following description, the data processing apparatus 12 is referred to as a “server”, and the headset-type terminal 314 is referred to as a “terminal”.

### Example 1

Since the flow of the specific processing is the same as that in Example 1 described in the first embodiment, a description thereof is omitted.

### Application Example 1

Since the flow of the specific processing is the same as that in Example 1 described in the first embodiment, a description thereof is omitted.

The specific processing unit 290 transmits a result of the specific processing to the headset-type terminal 314. In the headset-type terminal 314, the control unit 46A causes the speaker 240 and the display 343 to output the result of the specific processing. The microphone 238 acquires voice indicating a user input for the result of the specific processing. The control unit 46A transmits voice data indicating the user input acquired by the microphone 238 to the data processing apparatus 12. In the data processing apparatus 12, the specific processing unit 290 acquires the voice data.

The data generation model 58 is a so-called generative AI (Artificial Intelligence). An example of the data generation model 58 includes a generative AI such as ChatGPT (registered trademark) (Internet search <URL: https://openai.com/blog/chatgpt>). The data generation model 58 is obtained by causing a neural network to perform deep learning. A prompt including an instruction is input to the data generation model 58, and inference data such as voice data indicating voice, text data indicating text, and image data indicating an image (for example, still image data or moving image data) is input. The data generation model 58 infers the input inference data in accordance with the instruction indicated by the prompt, and outputs an inference result in one or more data formats among voice data, text data, image data, and the like. The data generation model 58 includes, for example, a text generation AI, an image generation AI, a multimodal generation AI, and the like. Here, inference refers to, for example, analysis, classification, prediction, and/or summarization, and the like. The specific processing unit 290 performs the above-described specific processing while using the data generation model 58. The data generation model 58 may be a model fine-tuned to output an inference result from a prompt that does not include an instruction, and in this case, the data generation model 58 can output an inference result from a prompt that does not include an instruction. In the data processing apparatus 12 and the like, a plurality of types of data generation models 58 are included, and the data generation model 58 includes AIs other than generative AI. Als other than generative AI are, for example, linear regression, logistic regression, a decision tree, a random forest, a support vector machine (SVM), k-means clustering, a convolutional neural network (CNN), a recurrent neural network (RNN), a generative adversarial network (GAN), or naive Bayes, and can perform various processes, but the present disclosure is not limited to such examples. Also, the AI may be an AI agent. Also, when the processing of each unit described above is performed by an AI, the processing is partially or entirely performed by the AI, but the present disclosure is not limited to such an example. Also, a process implemented by an AI including a generative AI may be replaced with a rule-based process, and a rule-based process may be replaced with a process implemented by an AI including a generative AI.

Also, the processing by the data processing system 10 described above is executed by the specific processing unit 290 of the data processing apparatus 12 or the control unit 46A of the smart device 14, but may also be executed by the specific processing unit 290 of the data processing apparatus 12 and the control unit 46A of the smart device 14. Also, the specific processing unit 290 of the data processing apparatus 12 acquires or collects information necessary for the processing from the smart device 14 or an external device, and the smart device 14 acquires or collects information necessary for the processing from the data processing apparatus 12 or an external device.

For example, a collection unit is realized by the control unit 46A of the smart device 14 or the specific processing unit 290 of the data processing apparatus 12. For example, an acquisition unit acquires step count data using the camera 42 or the communication I/F 44 of the smart device 14, and the data is processed by the specific processing unit 290 of the data processing apparatus 12. For example, an analysis unit is realized by the specific processing unit 290 of the data processing apparatus 12, and analyzes data from the collection unit and the acquisition unit. For example, a generation unit is realized by the specific processing unit 290 of the data processing apparatus 12, and generates a cooking menu using a generative AI. For example, a provision unit is realized by the output device 40 of the smart device 14 or the specific processing unit 290 of the data processing apparatus 12, and provides the generated cooking menu to a user. The correspondence relationship between each unit and the device or the control unit is not limited to the above-described example, and various changes are possible.

In the above embodiment, an example form in which the specific processing is performed by the data processing apparatus 12 has been described, but the technology of the present disclosure is not limited to this, and the specific processing may be performed by the headset-type terminal 314.

### Fourth Embodiment

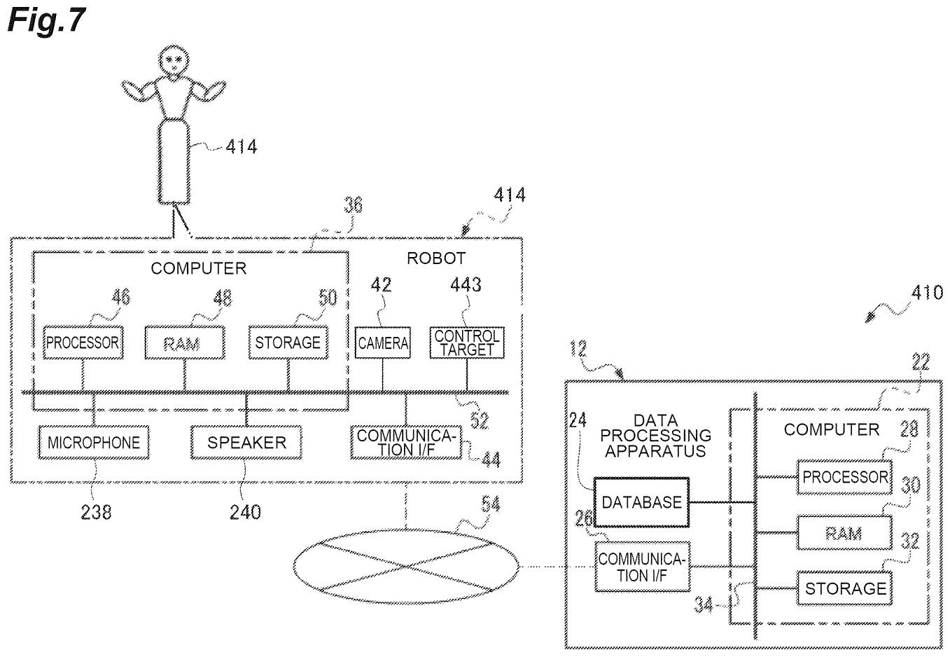

FIG. 7 illustrates an example of a configuration of a data processing system 410 according to a fourth embodiment.

As illustrated in FIG. 7, the data processing system 410 includes a data processing apparatus 12 and a robot 414. An example of the data processing apparatus 12 includes a server.

The data processing apparatus 12 includes a computer 22, a database 24, and a communication I/F 26. The computer 22 is an example of a “computer” according to the technology of the present disclosure. The computer 22 includes a processor 28, a RAM 30, and a storage 32. The processor 28, the RAM 30, and the storage 32 are connected to a bus 34. Also, the database 24 and the communication I/F 26 are also connected to the bus 34. The communication I/F 26 is connected to a network 54. An example of the network 54 includes a WAN (Wide Area Network) and/or a LAN (Local Area Network), and the like.

The robot 414 includes a computer 36, a microphone 238, a speaker 240, a camera 42, a communication I/F 44, and a control target 443. The computer 36 includes a processor 46, a RAM 48, and a storage 50. The processor 46, the RAM 48, and the storage 50 are connected to a bus 52. Also, the microphone 238, the speaker 240, the camera 42, and the control target 443 are also connected to the bus 52.

The microphone 238 receives an instruction or the like from a user 20 by receiving voice uttered by the user 20. The microphone 238 captures the voice uttered by the user 20, converts the captured voice into voice data, and outputs the voice data to the processor 46. The speaker 240 outputs voice in accordance with an instruction from the processor 46.

The camera 42 is a small digital camera on which an optical system such as a lens, a diaphragm, and a shutter, and an image sensor such as a CMOS (Complementary Metal-Oxide-Semiconductor) image sensor or a CCD (Charge Coupled Device) image sensor are mounted, and images the surroundings of the user 20 (for example, an imaging range defined by an angle of view corresponding to the width of the field of view of a general person with normal vision).

The communication I/F 44 is connected to the network 54. The communication I/Fs 44 and 26 manage exchange of various information between the processor 46 and the processor 28 via the network 54. The exchange of various information between the processor 46 and the processor 28 using the communication I/Fs 44 and 26 is performed in a secure state.

The control target 443 includes a display device, an LED of an eye part, and motors that drive an arm, a hand, a leg, and the like. The posture and gestures of the robot 414 are controlled by controlling the motors of the arm, hand, leg, and the like. A part of the emotions of the robot 414 can be expressed by controlling these motors. Also, the facial expression of the robot 414 can also be expressed by controlling the light emission state of the LED of the eye part of the robot 414.

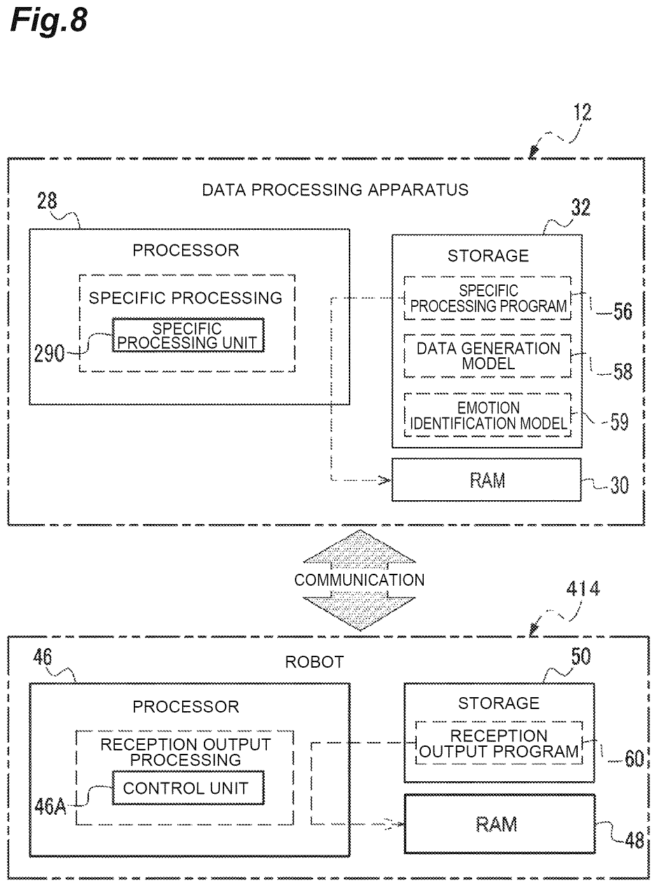

FIG. 8 illustrates an example of main functions of the data processing apparatus 12 and the robot 414. As illustrated in FIG. 8, in the data processing apparatus 12, specific processing is performed by the processor 28. A specific processing program 56 is stored in the storage 32.

The specific processing program 56 is an example of a “program” according to the technology of the present disclosure. The processor 28 reads the specific processing program 56 from the storage 32 and executes the read specific processing program 56 on the RAM 30. The specific processing is realized by the processor 28 operating as a specific processing unit 290 in accordance with the specific processing program 56 executed on the RAM 30.

A data generation model 58 and an emotion identification model 59 are stored in the storage 32. The data generation model 58 and the emotion identification model 59 are used by the specific processing unit 290.

In the robot 414, reception output processing is performed by the processor 46. A reception output program 60 is stored in the storage 50. The processor 46 reads the reception output program 60 from the storage 50 and executes the read reception output program 60 on the RAM 48. The reception output processing is realized by the processor 46 operating as a control unit 46A in accordance with the reception output program 60 executed on the RAM 48.

Next, specific processing by the specific processing unit 290 of the data processing apparatus 12 will be described. Each unit of the system described below is realized by the data processing apparatus 12 and the robot 414. In the following description, the data processing apparatus 12 is referred to as a “server”, and the robot 414 is referred to as a “terminal”.

### Example 1

Since the flow of the specific processing is the same as that in Example 1 described in the first embodiment, a description thereof is omitted.

### Application Example 1

Since the flow of the specific processing is the same as that in Example 1 described in the first embodiment, a description thereof is omitted.

The specific processing unit 290 transmits a result of the specific processing to the robot 414. In the robot 414, the control unit 46A causes the speaker 240 and the control target 443 to output the result of the specific processing. The microphone 238 acquires voice indicating a user input for the result of the specific processing. The control unit 46A transmits voice data indicating the user input acquired by the microphone 238 to the data processing apparatus 12. In the data processing apparatus 12, the specific processing unit 290 acquires the voice data.

The data generation model 58 is a so-called generative AI (Artificial Intelligence). An example of the data generation model 58 includes a generative AI such as ChatGPT (registered trademark) (Internet search <URL: https://openai.com/blog/chatgpt>). The data generation model 58 is obtained by causing a neural network to perform deep learning. A prompt including an instruction is input to the data generation model 58, and inference data such as voice data indicating voice, text data indicating text, and image data indicating an image (for example, still image data or moving image data) is input. The data generation model 58 infers the input inference data in accordance with the instruction indicated by the prompt, and outputs an inference result in one or more data formats among voice data, text data, image data, and the like. The data generation model 58 includes, for example, a text generation AI, an image generation AI, a multimodal generation AI, and the like. Here, inference refers to, for example, analysis, classification, prediction, and/or summarization, and the like. The specific processing unit 290 performs the above-described specific processing while using the data generation model 58. The data generation model 58 may be a model fine-tuned to output an inference result from a prompt that does not include an instruction, and in this case, the data generation model 58 can output an inference result from a prompt that does not include an instruction. In the data processing apparatus 12 and the like, a plurality of types of data generation models 58 are included, and the data generation model 58 includes AIs other than generative AI. AIs other than generative AI are, for example, linear regression, logistic regression, a decision tree, a random forest, a support vector machine (SVM), k-means clustering, a convolutional neural network (CNN), a recurrent neural network (RNN), a generative adversarial network (GAN), or naive Bayes, and can perform various processes, but the present disclosure is not limited to such examples. Also, the AI may be an AI agent. Also, when the processing of each unit described above is performed by an AI, the processing is partially or entirely performed by the AI, but the present disclosure is not limited to such an example. Also, a process implemented by an AI including a generative AI may be replaced with a rule-based process, and a rule-based process may be replaced with a process implemented by an AI including a generative AI.

Also, the processing by the data processing system 10 described above is executed by the specific processing unit 290 of the data processing apparatus 12 or the control unit 46A of the smart device 14, but may also be executed by the specific processing unit 290 of the data processing apparatus 12 and the control unit 46A of the smart device 14. Also, the specific processing unit 290 of the data processing apparatus 12 acquires or collects information necessary for the processing from the smart device 14 or an external device, and the smart device 14 acquires or collects information necessary for the processing from the data processing apparatus 12 or an external device.

For example, a collection unit is realized by the control unit 46A of the smart device 14 or the specific processing unit 290 of the data processing apparatus 12. For example, an acquisition unit acquires step count data using the camera 42 or the communication I/F 44 of the smart device 14, and the data is processed by the specific processing unit 290 of the data processing apparatus 12. For example, an analysis unit is realized by the specific processing unit 290 of the data processing apparatus 12, and analyzes data from the collection unit and the acquisition unit. For example, a generation unit is realized by the specific processing unit 290 of the data processing apparatus 12, and generates a cooking menu using a generative AI. For example, a provision unit is realized by the output device 40 of the smart device 14 or the specific processing unit 290 of the data processing apparatus 12, and provides the generated cooking menu to a user. The correspondence relationship between each unit and the device or the control unit is not limited to the above-described example, and various changes are possible.

In the above embodiment, an example form in which the specific processing is performed by the data processing apparatus 12 has been described, but the technology of the present disclosure is not limited to this, and the specific processing may be performed by the robot 414.

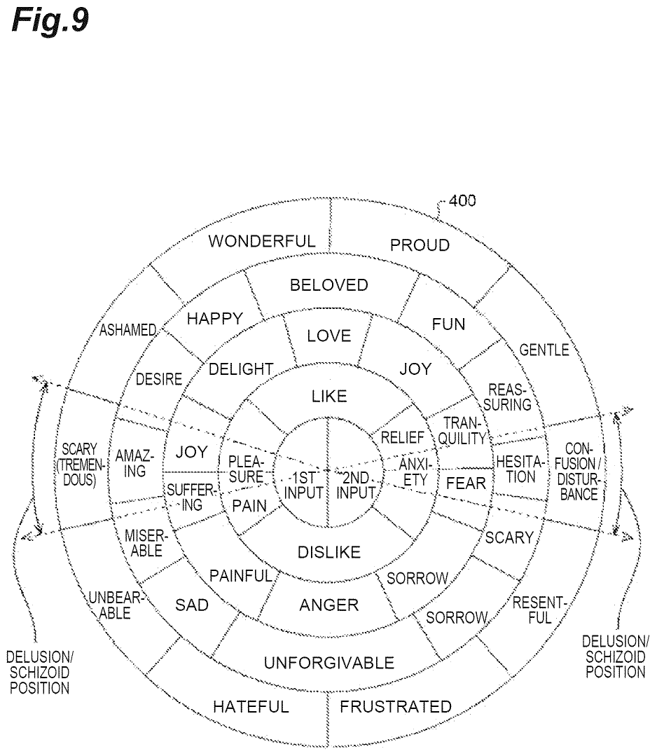

Note that the emotion identification model 59 as an emotion engine may determine a user's emotion according to a specific mapping. Specifically, the emotion identification model 59 may determine the user's emotion according to an emotion map (see FIG. 9), which is a specific mapping. Also, the emotion identification model 59 may similarly determine the robot's emotion, and the specific processing unit 290 may perform specific processing using the robot's emotion.

FIG. 9 is a diagram illustrating an emotion map 400 on which a plurality of emotions are mapped. In the emotion map 400, emotions are arranged in concentric circles radiating from the center. The closer to the center of the concentric circles, the more primitive the state of the emotion is arranged. On the outer side of the concentric circles, emotions representing states and actions arising from a state of mind are arranged. Emotion is a concept that also includes affect and mental states. On the left side of the concentric circles, emotions generated from reactions that generally occur in the brain are arranged. On the right side of the concentric circles, emotions that are generally induced by situational judgment are arranged. In the upward and downward directions of the concentric circles, emotions that are generated from reactions that generally occur in the brain and are induced by situational judgment are arranged. Also, on the upper side of the concentric circles, “pleasant” emotions are arranged, and on the lower side, “unpleasant” emotions are arranged. In this way, in the emotion map 400, a plurality of emotions are mapped based on the structure in which emotions are generated, and emotions that are likely to occur at the same time are mapped close to each other.

These emotions are distributed in the 3 o'clock direction of the emotion map 400, and usually go back and forth between relief and anxiety. In the right half of the emotion map 400, situational awareness is superior to internal sensations, resulting in a calm impression.

Since the inside of the emotion map 400 represents the inside of the mind and the outside of the emotion map 400 represents actions, the further one goes to the outside of the emotion map 400, the more visible (manifested in action) the emotion becomes.

Here, human emotions are based on various balances such as posture and blood sugar levels, and show a state of unpleasantness when those balances move away from the ideal, and a state of pleasantness when they approach the ideal. In robots, automobiles, motorcycles, and the like as well, emotions can be created based on various balances such as posture and remaining battery level, so as to show a state of unpleasantness when those balances move away from the ideal, and a state of pleasantness when they approach the ideal. The emotion map may be generated based on, for example, Dr. Mitsuyoshi's emotion map (Research on a speech emotion recognition and brain physiological signal analysis system of affect, Tokushima University, doctoral dissertation: https://ci.nii.ac.jp/naid/500000375379). In the left half of the emotion map, emotions belonging to a region called “reaction” where sensation is dominant are arranged. Also, in the right half of the emotion map, emotions belonging to a region called “situation” where situational awareness is dominant are arranged.

In the emotion map, two emotions that promote learning are defined. One is an emotion around the middle of negative “remorse” and “reflection” on the situation side. That is, it is when a negative emotion such as “I never want to feel this way again” or “I don't want to be scolded anymore” arises in the robot. The other is an emotion around positive “desire” on the reaction side. That is, it is when there is a positive feeling such as “I want more” or “I want to know more”.

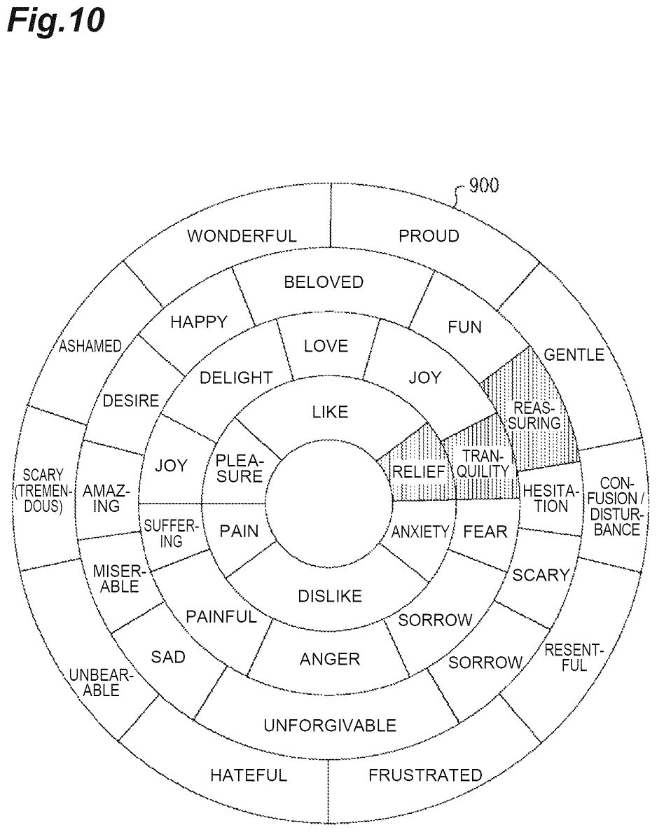

The emotion identification model 59 inputs a user input into a pre-trained neural network, acquires an emotion value indicating each emotion shown in the emotion map 400, and determines the user's emotion. This neural network is pre-trained based on a plurality of learning data that are combinations of user inputs and emotion values indicating each emotion shown in the emotion map 400. Also, this neural network is trained such that emotions arranged close to each other have close values, as in the emotion map 900 shown in FIG. 10. FIG. 10 shows an example in which a plurality of emotions, “relief,” “peace of mind,” and “reassured,” have close emotion values.

Although the system according to the present disclosure has been described above mainly with respect to the functions of the data processing apparatus 12, the system according to the present disclosure is not necessarily implemented in a server. The system according to the present disclosure may be implemented as a general information processing system. The present disclosure may be implemented as, for example, a software program that runs on a personal computer, or an application that runs on a smartphone or the like. The method according to the present disclosure may be provided to a user in a Saas (Software as a Service) format.

In the above embodiment, an example form in which the specific processing is performed by one computer 22 has been described, but the technology of the present disclosure is not limited to this, and distributed processing for the specific processing may be performed by a plurality of computers including the computer 22. For example, the data generation model 58 may be provided in an external device of the data processing apparatus 12, and the external device may generate data according to the input data.

In the above embodiment, an example form in which the specific processing program 56 is stored in the storage 32 has been described, but the technology of the present disclosure is not limited to this. For example, the specific processing program 56 may be stored in a portable computer-readable non-transitory storage medium such as a USB (Universal Serial Bus) memory. The specific processing program 56 stored in the non-transitory storage medium is installed in the computer 22 of the data processing apparatus 12. The processor 28 executes the specific processing according to the specific processing program 56.

Also, the specific processing program 56 may be stored in a storage device such as a server connected to the data processing apparatus 12 via the network 54, and the specific processing program 56 may be downloaded in response to a request from the data processing apparatus 12 and installed in the computer 22.

Note that it is not necessary to store all of the specific processing program 56 in a storage device such as a server connected to the data processing apparatus 12 via the network 54, or to store all of the specific processing program 56 in the storage 32, and a part of the specific processing program 56 may be stored.

As hardware resources for executing the specific processing, various processors shown below can be used. Examples of the processor include a CPU, which is a general-purpose processor that functions as a hardware resource for executing the specific processing by executing software, that is, a program. Also, examples of the processor include a dedicated electric circuit, which is a processor having a circuit configuration specifically designed to execute specific processing, such as an FPGA (Field-Programmable Gate Array), a PLD (Programmable Logic Device), or an ASIC (Application Specific Integrated Circuit). A memory is built in or connected to any of the processors, and any of the processors executes the specific processing by using the memory.

The hardware resource that executes the specific processing may be configured by one of these various processors, or may be configured by a combination of two or more processors of the same type or different types (for example, a combination of a plurality of FPGAs, or a combination of a CPU and an FPGA). Also, the hardware resource that executes the specific processing may be one processor.

As an example of a configuration with one processor, first, there is a form in which one processor is configured by a combination of one or more CPUs and software, and this processor functions as a hardware resource for executing the specific processing. Second, there is a form in which a processor that realizes the functions of an entire system including a plurality of hardware resources for executing the specific processing with one IC chip, as represented by an SoC (System-on-a-chip) or the like, is used. In this way, the specific processing is realized using one or more of the various processors described above as hardware resources.

Furthermore, as a hardware structure of these various processors, more specifically, an electric circuit in which circuit elements such as semiconductor elements are combined can be used. Also, the specific processing described above is merely an example. Therefore, it goes without saying that unnecessary steps may be deleted, new steps may be added, or the processing order may be changed within a scope that does not depart from the gist.

The description and illustrations shown above are detailed descriptions of the parts related to the technology of the present disclosure, and are merely an example of the technology of the present disclosure. For example, the description regarding the above-described configuration, function, operation, and effect is a description regarding an example of the configuration, function, operation, and effect of the part related to the technology of the present disclosure. Therefore, it goes without saying that unnecessary parts may be deleted, new elements may be added, or replacements may be made to the description and illustrations shown above within a scope that does not depart from the gist of the technology of the present disclosure. Also, in order to avoid confusion and facilitate understanding of the parts related to the technology of the present disclosure, in the description and illustrations shown above, descriptions regarding common general technical knowledge and the like that do not require particular explanation for enabling the implementation of the technology of the present disclosure are omitted.

All documents, patent applications, and technical standards described in this specification are incorporated herein by reference to the same extent as if each individual document, patent application, and technical standard were specifically and individually indicated to be incorporated by reference.

Regarding the above embodiments, the following is further disclosed.

### Application Example 1

A system comprising a generative AI unit, a scent blending unit, and a user interface unit. The generative AI unit analyzes video or music data input from a content distribution platform and determines scent components based on the scene or music atmosphere using video analysis technology and music analysis technology. The scent blending unit selects necessary fragrance materials from a plurality of fragrance material cartridges based on instructions from the generative AI unit, and performs blending with precise control. The user interface unit provides a means for a user to adjust the intensity and duration of the scent, and can accept text or voice input. This is a system that allows a user to experience content through the three senses of sight, hearing, and smell, and significantly improves the immersive experience of the content.

The system, wherein the generative AI unit is provided with video analysis technology for recognizing scenes and objects in a video and extracting scent elements based thereon, and for example, when a sea scene is displayed, recognizes elements such as “salt,” “water,” and “algae,” and imagines a related scent. This makes it possible to generate a scent in real time according to the content of the video and provide it to the user.

The system, wherein the scent blending unit is provided with fragrance material cartridges, includes pumps and valves for adjusting the type and amount of each fragrance material, and precisely controls and blends the fragrance materials based on instructions from the generative AI unit. This makes it possible to accurately reproduce the scent desired by the user on the spot and to provide a personalized scent experience.

A system for blending a fragrance, comprising: circuitry and a database, wherein the circuitry is configured to: receive input of a scent image from a user; analyze the scent image input from the user and extract related scent keywords using natural language processing technology; and blend a plurality of fragrances based on the keywords to generate a scent corresponding to the keywords, wherein the database stores correspondence relationships between the keywords related to the scent and the fragrances, and wherein the circuitry refers to the database when determining components of the scent.

The system, wherein the input is text input or voice input.

The system, wherein the circuitry is configured to: receive video data as the input; and recognize scenes or objects in the video data to blend and release in real time the scent imagined from the video data.

The system, wherein the circuitry analyzes elements in the video data and determines scent components based on the elements in the video data.

The system, wherein the system comprises: a fragrance cartridge; and a pump and a valve for controlling a type and amount of the fragrance.

A method for blending a fragrance, the method comprising: receiving input of a scent image from a user; and analyzing the scent image input from the user and extracting related scent keywords using natural language processing technology; blending a plurality of fragrances based on the keywords to generate a scent corresponding to the keywords; and referring to a database when determining components of the scent, wherein the database stores correspondence relationships between the keywords related to the scent and fragrances.

1. A system for blending a fragrance, comprising:
circuitry and a database,
wherein the circuitry is configured to:
receive input of a scent image from a user;
analyze the scent image input from the user and extract related scent keywords using natural language processing technology; and
blend a plurality of fragrances based on the keywords to generate a scent corresponding to the keywords,
wherein the database stores correspondence relationships between the keywords related to the scent and the fragrances, and
wherein the circuitry refers to the database when determining components of the scent.

2. The system according to claim 1, wherein the input is text input or voice input.

3. The system according to claim 1,
wherein the circuitry is configured to:
receive video data as the input; and
recognize scenes or objects in the video data to blend and release in real time the scent imagined from the video data.

4. The system according to claim 3,
wherein the circuitry analyzes elements in the video data and determines scent components based on the elements in the video data.

5. The system according to claim 1,
wherein the system comprises:
a fragrance cartridge; and
a pump and a valve for controlling a type and amount of the fragrance.

6. A method for blending a fragrance, the method comprising:
receiving input of a scent image from a user; and
analyzing the scent image input from the user and extracting related scent keywords using natural language processing technology;
blending a plurality of fragrances based on the keywords to generate a scent corresponding to the keywords; and
referring to a database when determining components of the scent, wherein the database stores correspondence relationships between the keywords related to the scent and fragrances.
